(19) United States
(12) Patent Application Publication
(10) Pub. No.: US 2008/0019913 A1
(43) Pub. Date: Jan. 24, 2008

(54) ENKEPAHLIN ANALOGS WITH IMPROVED BIOAVAILABILITY

(75) Inventors: Robin L. Polt, Tucson, AZ (US); Edward J. Bilsky, Biddeford Pool, ME (US)

Correspondence Address: OBLON, SPIVAK, MCCLELLAND MAIER & NEUSTADT, P.C., 1940 DUKE STREET, ALEXANDRIA, VA, 22314, US

(73) Assignee: University of Arizona Office of Technology Transfer, Tucson, AZ (US)

(21) Appl. No.: 11/775,045

(22) Filed: Jul. 9, 2007

Related U.S. Application Data
(60) Provisional application No. 60/806,751, filed on Jul. 7, 2006.
(60) Provisional application No. 60/819,428, filed on Jul. 7, 2006.

(51) Int. Cl. A61K 38/08; A61K 49/00; C07K 7/00; G01N 33/53

(52) U.S. Cl. 424/9.2; 435/7.2; 514/17; 530/330

(57) ABSTRACT

Peptides having improved bioavailability, especially analogs of enkephalins, with biousian properties which have two conformations or two conformation ensembles with different solubility properties.

### CROSS-REFERENCE TO RELATED APPLICATIONS

This application claims priority to U.S. Provisional Application Nos. 60/819,428 and 60/806,751, both filed Jul. 7, 2006, and to U.S. Provisional Application No. 60/______, “Glycopeptide Targets”, filed on Jun. 22, 2007, each of which is hereby incorporated by reference in its entirety.

### STATEMENT REGARDING FEDERALLY SPONSORED RESEARCH OR DEVELOPMENT

This invention was made with government support under Grants 14-02-01-0471 and 14-05-1-0807 awarded by the Office of Naval Research and by Grant CHE-607917 awarded by the National Science Foundation. The government has certain rights in the invention.

### BACKGROUND OF THE INVENTION

1. Field of the Invention

Enkephalins, such as Met- or Leu enkephalin, and enkephalin-like molecules (enkepahlin analogs) like DAMGO, play a role in regulating pain or nociception. These molecules interact with the mu opioid receptor which also recognizes opioid alkaloids like codeine and morphine. [Met]-enkephalin and [Leu]-enkephalin have the following structures: Tyr-Gly-Gly-Phe-Met (SEQ ID NO: 1) and Tyr-Gly-Gly-Phe-Leu (SEQ ID NO: 2).

DAMGO [D-Ala²,Me-Phe⁴Gly⁵(ol)]enkephalin is an enkephalin analog that selectively binds to the mu opioid receptor. DAMGO is often used as a model molecule in mu opioid experiments. Enkephalin peptide analogs based on DAMGO having improved bioavailability have been produced by modifying physiologically active peptides and glycopeptides to include a hydrophilic peptide address segment. Amphipathic DAMGO-like or enkephalin-like peptides or glycopeptides exhibit biousian properties providing enhanced transcellular delivery of these agents, for example, through the blood-brain barrier. Pharmacological methods of using these peptide analogs, including provision of antinociceptive effects.

2. Description of the Related Art

The discovery of endogenous opioid ligands (1) and their receptors (2) gave an early impetus to the concept of drugs for the central nervous system (CNS) based on peptide neurotransmitters. In principle, peptides could replace naturally occurring alkaloids such as morphine or codeine, or petroleum-derived drugs such as fentanyl. Unfortunately, this impetus was quickly dampened as pharmacologists encountered the many problems associated with the synthesis (3), binding (4), stability (5), and biodistribution (6) of peptides. Over the intervening decades, many of these problems have been addressed, and the prospects for peptide-based drugs once again seem bright (7).

One of the last problems to be addressed is the penetration of the blood-brain barrier (BBB; 8). Typical peptide neurotransmitters have MWs>500, log Ps<−2.0, and many more H-bonds than Lipinski's rules of transport would allow (9). In fact, the very features that make peptides useful as neurotransmitters make them violate nearly all of the transport rules for typical pharmaceutical compounds. Neurotransmitters, such as enkephalins and DAMGO, possess highly amphipathic conformations that promote strong interactions with membranes^{12, 13 14}. Typically, [Met]- or [Leu]-enkephalin is released from a presynaptic vesicle and is required to travel the distance between the pre- and post-synaptic membrane (several hundred angstroms at most) before it is strongly adsorbed to the post-synaptic neuronal membrane. Binding of the neurotransmitter peptide (e.g., [Met]- or [Leu]-enkephalin) to the post-synaptic neuronal membrane faciliates its subsequent rapid binding to a membrane-bound opioid receptor as shown in FIG. 1.

Enkephalins and DAMGO are rapidly bound to the post-synaptic membrane and these binding properties correlate with their poor ability to cross the blood brain barrier. Previous enkephalin analogs, including DAMGO are not useful as drugs since they have such a short half-life in serum, largely due to their afinity for membranes, which prevents them from displaying useful biodistribution properties. Previous attempts to make enkephalins more lipophilic to penetrate the blood-brain barrier via difusion have proved ineffective since this limits aqueous solubility of the enkephalin analogs.

The inventors' studies with glycosylated enkephalins have given rise to the concepts of membrane hopping (10) and the biousian hypothesis (11). As shown below, the inventors have developed enkephalin analogs based on the DAMGO structure which exhibit biousian behavior, such as an ability to membrane hop, and which have enhanced pharmacological and transport properties compared to native enkephalins and enkephalin analogs like DAMGO.

### BRIEF SUMMARY OF THE INVENTION

A DAMGO ([D-Ala²,Me-Phe⁴Gly⁵(ol)]enkephalin) analog which contains modifications of one, two, three, four or more chemical moieties in DAMGO shown by formula (I) below:

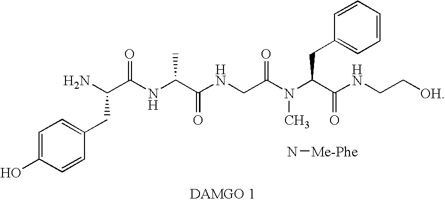

The moieties which may be modified are any of those shown in the above structure, including —OH, NH2, —NH—, C═O, methyl or phenyl.

To provide biousian properties, preferably, a DAMGO analog may contain (or be substituted by) one or more additional hydrophilic moieties compared to DAMGO. Such moieities include, but are not limited to a sugar or carbohydrate group, phosphate, pyrophosphate, phosphonate, cholate, sulfate, or sulfonate groups. DAMGO may also be modified by addition of an alkyl, cycloalkyl or aryl group containing at least one hydrophilic substituent and optionally other substituents.

For example, a DAMGO analog may contain a hydrophilic group at the C-terminus instead of unsubstituted glycinol found in DAMGO. Furthermore, the hydroxyl group on the glycinol moiety of DAMGO may be substituted with a more hydrophilic group that increases the hydrophilicity of the DAMGO analog compared to DAMGO. The glycinol moiety on DAMGO may replaced with an amino acid residue or modified amino acid residue more hydrophilic than glycinol. For example, the glycinol moiety can be replaced by L-serine amide, L-serine amide β-D-xyloside, L-serine amide β-D-glucoside, or L-serine amide β-lactoside.

Other DAMGO analogs of the invention comprise the following structure:

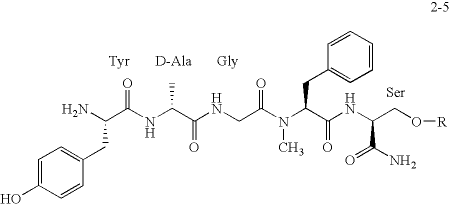

where R is hydrogen or a hydrophilic group. This structure contains a carboxyamino group near the C-terminus. The R group may be hydrogen, as is found in DAMGO, or may represent a hydrophilic group, such as a sugar or carbohydrate group. R may also comprise a phosphate, pyrophosphate, phosphonate, cholate, sulfate, or sulfonate group, and these groups may be further substituted with alkyl, cycloalkyl or aryl groups which may have additional substituents.

The DAMGO analogs of the invention may have a structure in which DAMGO is modified at one or more of the sites or moieties substituted in the structures shown below. These DAMGO analogs have the following structures (respectively, LYM-1311 and its TFA salt, and LYM-1312 and its TFA salt):

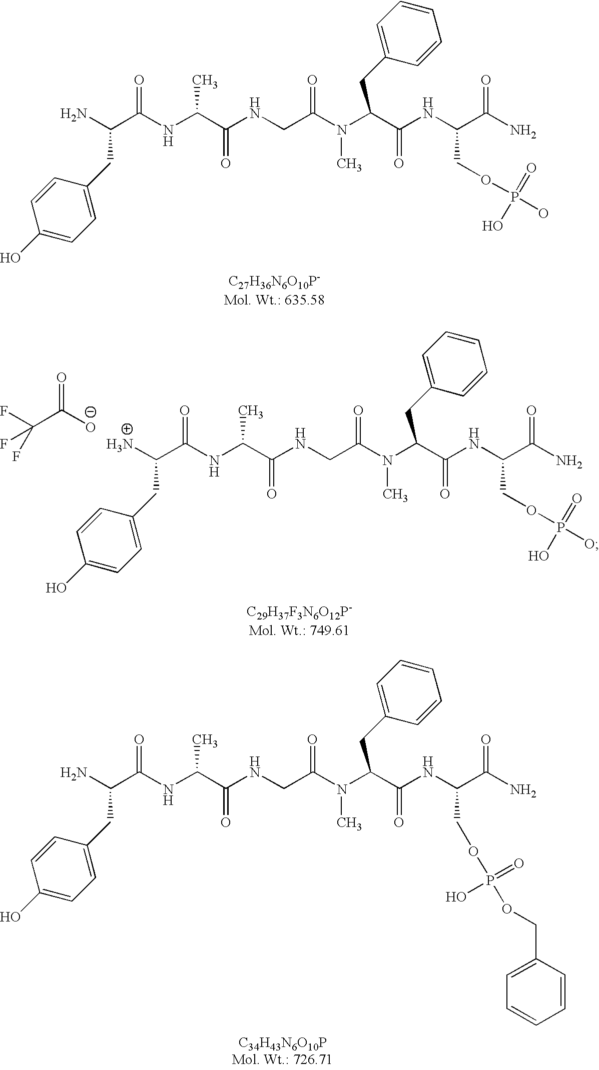

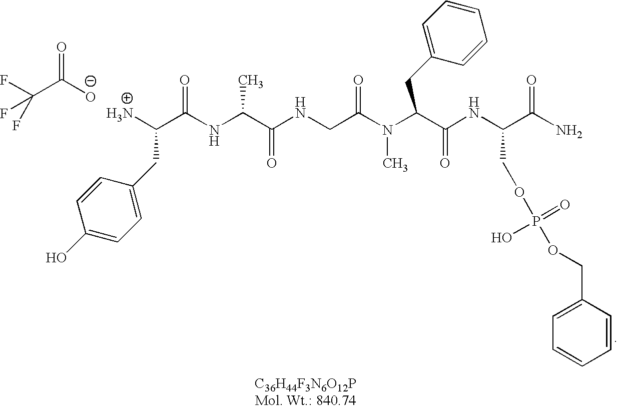

The antinociceptive activities of these compounds (LYM-1311 and LYM-1312) are shown in FIG. 7.

Other DAMGO analogs according to the invention will have a degree of glycosylation that will range from about 1.25 to 1.75 in terns of hydrodynamic glucose units or range between 0.75 to 0.90 g.u. in terms of surface-derived amphipathicity values.

DAMGO analogs according to the invention may have molecular masses of about 1,000, 2,000, 3,000 or more Da. Generally, DAMGO analogs with lower molecular masses are preferred, but those with higher molecular masses may still have desirable properties, including an ability to cross the blood brain barrier when suitably modified, e.g., by appropriate hydrophilic groups. Thus, many DAMGO analogs will be about the mass of a peptide having about 5-7 amino acid residues or have masses about 1,000 Da.

DAMGO analogs may contain additional amino acid residues not present in DAMGO and may comprise, for example, up to 7, 8, 9, 10, 11, 12, 13, 14, 15 or 16 D- or L-amino acid residues or mixtures of both D and L amino acid residues. Longer DAMGO analogs provided with the appropriate hydrophilic properties, such as glycosylation can penetrate the blood brain barrier. 1, 2, 3 or 4 amino acid residues of DAMGO (shown above) may be substituted with other amino acids. Amino acids for substitution may be selected based on similarity of charge or side-chain size.

Analogs may be designed in the form of prodrugs or conjugates with other molecules which are metabolized into biologically active derivatives when administered. Methods for design and use of prodrugs are well-known in the art and such methods are incorporated by reference to Remington, Science and Practice of Pharmacy, 21rst edition, especially page 958 and to citations 99-104 cited on that page. The DAMGO analogs may also be modified or formulated as compositions as described in Part 5 of Remington, The Science and Practice of Pharmacy, 21rst edition, which is incorporated by reference.

The DAMGO analogs of the invention may be mu opioid agonists, inverse agonists or antagonists. For example, a DAMGO analog may have lower binding affinity for the post-synaptic membrane than DAMGO or may have a lower binding affinity for opioid mu receptor than DAMGO, or both. Unlike natural enkephalins or DAMGO itself, containing Class L amphipathic helices that lead to intracellular peptide delivery, e.g., to the post-synaptic membrane, a DAMGO analog need not irreversibly bind to the post-synaptic membrane and preferably does not, so as to promote membrane hopping. For example, a DAMGO analog may adopt a Class A type helical structure that permits it to membrane-hop and promotes its ability to be delivered transcellularly. Modification of the helical properties of a DAMGO analog permit modulation or balancing of its ability to be delivered intracellularly and transcellularly.

A DAMGO analog will preferably have altered properties with regard to penetration of the blood brain barrier (BBB) compared to DAMGO. For example, it may penetrate the blood brain barrier faster or to a greater extent than DAMGO. Alternatively, it may cross this barrier more slowly or to a lesser extent than DAMGO. DAMGO analogs that do not easily cross the blood-brain barrier exhibit few if any central nervous system side effects and may be used to treat diseases modulated by peripheral opioid receptors, such as gastrointestinal diseases or disorders. A DAMGO analog may contain modifications to increase its biological half-life, biological adsorption, or passage across the blood brain barrier.

Most preferably, the DAMGO derivatives of the invention will not cross cellular membranes to enter the cytoplasm of cells, as may be the case with analogs having lipophilic modifications. Thus, even though the DAMGO derivatives enter cells upon endocytosis, they remain encapsulated within a membrane barrier, and are topologically outside the cellular barrier (e.g. the capillary endothelium). Upon exocytosis, the DAMGO derivatives are delivered to the luminal face of the cellular barrier without exposure to the degrading enzymes within the cytoplasm.

Pharmaceutical compositions comprising the DAMGO analogs of the invention are also contemplated. These will generally contain at least one pharmaceutically acceptable carrier or excipient and may contain other pharmaceutically active ingredients in addition to the DAMGO analog. Ways of compounding molecules like DAMGO are well-known and are also incorporated by reference to Remington, The Science and Practice of Pharmacy, 21rst edition, Part 2. A pharmaceutical composition containing a DAMGO analog may be in the form of a solution, emulsion, suspension, liposome or lipid bilayer. The DAMGO analog may be modified with groups, that facilitate its compartmentalization into a lipid phase (e.g., a lipid tail) or hydrophilic phase of a liposome or lipid bilayer. DAMGO analogs may also be conjugated to detectable markers or substrates.

The DAMGO analogs of the invention may be administered to subjects for relieving or modulating pain or for inducing analgesia or sedation. Those with skill in the pharmaceutical arts can easily determine appropriate amounts of the analog to administer for a particular effect based on the analog's specific properties, such as its biological half-life, its ability to cross the blood brain barrier, etc. The properties of opioid drugs are well-known as are the disorders or diseases for which they are perscribed and are also incorporated by reference to Chapter 83 of Remington, The Science and Practice of Pharmacy, 21rst edition.

The invention also involves the treatment of diseases or disorders mediated via the mu opioid receptor which is recognized by a DAMGO analog. These include methods for reducing blood pressure, respiration, or decreasing bowel motility, or methods for modulating itching, nausea, euphoria, or miosis (constricted pupils). The DAMGO analog may be administered by conventional routes, including topical, oral and parenteral routes described below.

Screening parameters useful in the identification of biousian molecules include, but are not limited to: reversed-phase HPLC retention times; Langmuir isotherms to lipophilic surfaces; data from surface plasmon resonance (SPR) studies; vesicle binding studies using microcalorimetry; binding to micelles and bicelles using nuclear magnetic resonance (NMR) or circular dichroism (CD) methods.

A molecule is “biousian” if it has two conformations or two confornational ensembles that have different solubility properties. For example, if a peptide can fold in such a way as to make it water soluble, and can fold in another way to make it more lipid soluble, it would be called biousian.

An “isolated” molecule or DAMGO analog is one that is substantially free of the materials with which it is associated in its native or synthetic environment. By substantially free, is meant at least 50%, preferably at least 70%, more preferably at least 80%, and even more preferably at least 90, 95, or 99% free of these materials.

“Administration” includes any method of introducing the compositions of the present invention into a subject or exposing a subject to these compositions. This includes administration of prodrugs which convert into a compound of the invention when administered to a subject or which are otherwise treated to release an active form prior to administration. The DAMGO analogs of the invention may be administered by a parenteral, oral, or topical route. Specifically, these routes include intravenous (i.v.), intradermal, subcutaneous (s.c.), intracerebral, intracerebroventricularal (i.c. v.), intrathecal, peridural, transmucosal, transdernal, inhalational (e.g., intratracheal, intrapulmonary, or intrabroncial), intransal, oral, subuccal, transdermal, and rectal administration.

A “pharmaceutically acceptable carrier” includes any and all carriers and excipients such as solvents, dispersing agents, emulsions, lipid bilayers, liposomes, coatings, antibacterial or antifungal agents, isotonic agents, pH buffers, and absorption modulating agents, and the like, compatible with a DAMGO analog and suitable for pharmaceutical administration. The use of such carriers, excipients and agents for administration of pharmaceutically active substances is well known in the art, but is also incorporated by reference to Remington, The Science and Practice of Pharmacy, 21^{st} edition (2005) and to The Handbook of Pharmaceutical Excipients, 3^{rd} edition (2000).

### BRIEF DESCRIPTION OF THE DRAWINGS

FIG. 1 depicts the effects of glycosylation on peptide neurotransmitters. Once released the peptide may assume an active or inactive conformation. In its active conformation, once bound to the post-synaptic membrane, the peptide is rapidly associated with the mu receptor. In its inactive form, the peptide may bind also bind to the post-synaptic membrane, but does not associate with the mu receptor. The peptide in its inactive conformation may diffuse or be transported to other membranes, where it may assume an active conformation and associate with a mu receptor. Peptide neurotransmitters strongly associate with membranes. (A, above) After release from the presynaptic neuron, peptide neurotransmitters (e.g. enkephalins) strongly associate with the postsynapticmembrane(kₒₙ>>k_{off}) and bind to a G-protein coupled receptor (μ-opioid receptor or MOR) via a membrane-bound conformation (Fisher's lock & key). Previous studies show that active conformations are favored in the membrane and inactive conformations are favored in the absence of a membrane. Incorporation of glycosides to optimize membrane hopping. (B, below) Incorporation of a glycoside moiety, represented by the 270°, 180°, 120° and 90° arcs, shifts the kₒₙ/k_{off} equilibrium to facilitate ‘membrane hopping’. Drug-like activity results when the kₒₙ/k_{off} ratio is near ideal. Previous studies show that membrane-bound (active) conformations of glycosylated enkephalins differ from their aqueous (inactive) conformations (1, 2).

FIG. 2 depicts the energetics of aqueous vs. membrane-bound states of the glycosylated peptide. Increasing the number of carbohydrate residues may decrease the energy of aqueous ensemble (1, 2, 3, 4, 5 . . . ) without substantially affecting the membrane bound ensemble (A, B, C . . . ).

FIG. 3 shows the physical structure of DAMGO, compound (1), and DAMGO analogs—compounds (2-5). Increasing water-solubility is introduced via the carboxyamide group (2), carboxamide+β-xyloside (3), carboxamide+β-glucoside (4), and carboxamide+β-lactoside (5) that are added at the right end of DAMGO. The peptide “message” is represented by Tyr-D-Ala-Gly-(N-Me)Phe. The ethanolamide(glycinol) has been assigned a hydrodynamic value of 0.25 glucose units (“g.u.”), carboxamide group assigned a value of 2 g.u., and the saccharides of (3, 4 and 5) the accepted values of 0.5, 1, and 2 g.u., respectively.

FIG. 4 depicts the calculated Connolly Surfaces for the μ-selective DAMGO peptide series of DAMGO (1) and DAMGO analogs (2-5). The Connolly Surface, calculated in Å², was divided into two sections, a lipophilic surface (blue), A_{lipid}, associated with the peptide message segment YaG(N-Me-F), and a hydrophilic surface (red), A_{water}, defined by the address segment.

FIG. 5 graphs the centrally-mediated analgesia or antinociception. A U-shaped or V-shaped curve is correlated with the two different measures of amphipathicity. The hydrodynamic values (glucose units, g.u.) or Connolly-derived amphipathicity values are plotted along the X-axes, and A₅₀ values (and 95% confidence intervals) derived from mouse i.v. tail-flick data are plotted on the Y-axes. Both analyses produce a U-shaped or V-shape, consistent with the biousian hypothesis¹⁰. The amphipathicity values, A, were calculated using the formula A=e^{-Awater/Alipid}, where A_{water}=the Connolly surface area of the hydrophilic moiety (Å²) and A_{lipid}=the Connolly surface area of the rest of the lipophilic peptide message segment YaG(N-MeF).

FIG. 6 shows the Intravenous (i.v.) versus intracerebroventricular potencies of DAMGO, compound (1), and its analogs, compounds (2-5). The analgesic potencies (A₅₀ values and 95% confidence intervals) are measured in the mouse 55° C. tail-flick assay after i.c.v. administration (horizontal axis, nmoles per mouse), and i.v. administration (vertical axis, μmoles per kg). Morphine sulfate (a μ-agonist) is shown as a reference point, but has been excluded from the correlation values.

FIG. 7A, B, C and D show the antinociceptive effects (mean % nociception) of administering various dosages of DAMGO analogs LYM-1311 or LYM-1312. Panels A and B show results of i.c.v. administration and lower panels C and D show results of i.v. administration.

The patent or application file contains at least one drawing executed in color. Copies of this patent or patent application publication with color drawing(s) will be provided by the office upon request and payment of the necessary fee.

### DETAILED DESCRIPTION OF THE INVENTION

The inventors have discovered that by making a DAMGO analog more hydrophilic, e.g., by glycosylation, improves the central effects of the resulting DAMGO related molecules or analogs. This discovery was based on production of a series of μ-agonist DAMGO analogs that were synthesized and pharmacologically characterize in accord with the biousian hypothesis of membrane hopping.

DAMGO was altered by incorporating moieties of increasing water solubility into its C-terminus via carboxamide and simple glycoside additions. The hydrophilic C-terminal moieties were varied from glycinol in DAMGO, compound (1), to L-serine amide in compound (2), L-serine amide b-D-xyloside in compound (3), L-serine amide b-D-glucoside in compound (4), and finally to L-serine amide b-lactoside in compound (5).

Opioid binding and mouse tail-flick studies were performed to assess functional activity. Antinociceptive potency (intravenous) increased, passing through a maximum (A₅₀≈0.2 μmol/kg) for 2 and 3 as membrane affinity versus water solubility became optimal, and dropped off (A₅₀≈1.0 μmol/kg) for compounds (4) and (5) as water solubility dominated molecular behavior.

Intravenous A₅₀ values were plotted versus hydrodynamic values (glucose units, g.u.) for the glycoside moieties, or the hydrophilic/hydrophobic Connolly surface areas (A₅₀ versus e^{-Awater/Alipid}), and provided either a V-shaped or a U-shaped curve, as predicted by the biousian hypothesis. The μ-selective receptor profile was maintained (Ki's=0.66-1.3 nM) upon modifications at the C-terminus. Based on these data, the optimal degree of glycosylation for the DAMGO peptide message in the tested compounds was determined to be between 1.25 and 1.75 g.u. (hydrodynamic g.u.), or between 0.75 and 0.90 in terms of the surface-derived amphipathicity values.

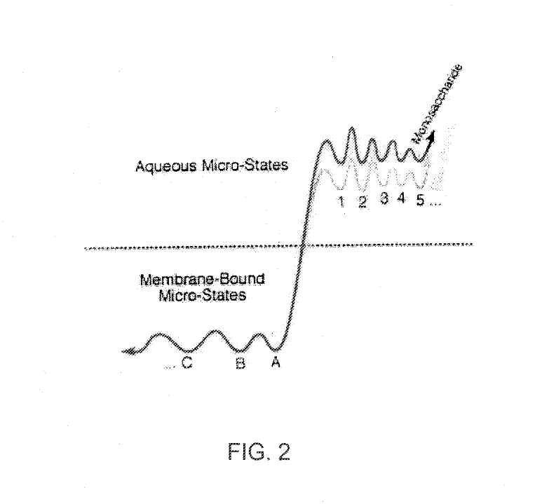

It was found that the attachment of a glycoside or another water-soluble moiety (e.g. a cationic amino acid residue, such as the arginine in TAPA, Tyr-D-Arg-Phe-beta-Ala-OH; 14) in the appropriate position can lead to increased stability of the aqueous state without perturbing the membrane-bound conformation of the peptide message. Thus, instead of simply binding to a biologic membrane, a DAMGO analog glycopeptide can ‘hop off’ the membrane, free to travel some distance before it encounters another membrane that will permit the glycopeptide to ‘hop on’ again. By carefully balancing the free energy of the two states (e.g. membrane-bound state versus aqueous state, FIG. 2), the optimal amount of time will be spent on the membrane for binding and endocytosis versus the amount of time spent moving through the aqueous compartments in vivo. This is the heart of the biousian hypothesis: two ‘ousia’ (essences or substances) potentially exist within the same molecule. In this way, peptide neurotransmitters can be converted from compounds that spend most of their time associated with membranes into drug-like molecules that more freely diffuse throughout the aqueous compartments of the biologic organism.

Methods and Materials

To further explore and exploit the biousian hypothesis, the classical μ-selective agonist DAMGO (1;15) was used as a lipophilic peptide ‘message’ (16). It is generally agreed that the μ-receptor is responsible for the bulk of the antinociceptive effects of opioid agonists and as the majority of opioid analgesics that have been studied or used clinically are μ-agonists. Thus, the biousian properties of DAMGO analogs were investigated in the context of a pure μ-agonist.

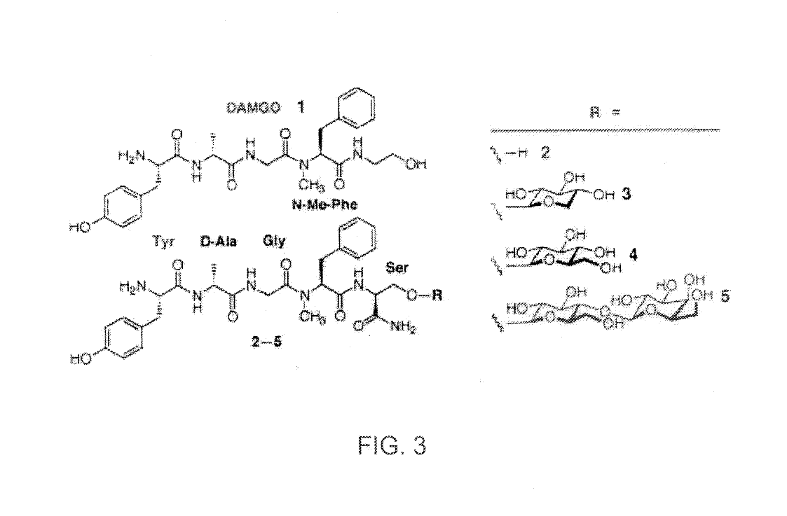

Modification of the parent peptide 1 (DAMGO) included the addition of moieties that increased the water-soluble ‘address’ segment of the molecule (FIG. 3). Peptide 2 and glycopeptides 3-5 were synthesized using published Fmoc methods with O)(1H-benzotriazole-1yl)-N,N,N′,N-tetramethyluronium hexafluorophosphate/diisopropylethylamine (HBTU/DIEA) and Rink amide resin on a Protein Technologies, Inc. (Tucson, Ariz., USA) PS3 synthesizer (3). FIG. 3 depicts DAMGO (1) and compounds (2-5).

Radioligand-Binding Studies

Binding was determined in Chinese hamster ovary (CHO) cell membranes expressing either the human μ-, Δ-, or κ-opioid receptors (MOR, DOR, and KOR). Cells were incubated with 12 concentrations of glycopeptide and the indicated radiolabeled ligand (Table 1, below). Non-specific binding was measured by inclusion of 10 μM naloxone. Data are the mean Kᵢ values±SEM from three experiments performed in triplicate.

Animal and Injections

All in vivo studies used adult male ICR mice (25-30 g; Harlan Industries, Cleveland, Ohio, USA) that were maintained on a 12 h light/dark cycle (lights on at 07:00 hours) in a temperature and humidity-controlled animal colony. All testing was carried out between 10:00 and 15:00 hours. Studies were carried out in accordance with the Guide for the Care and Use of Laboratory Animals as adopted and promulgated by the National Institutes of Health.

For intracerebroventricular (i.c.v.) injections, mice were lightly anesthetized with ether and an incision was made in the scalp. Injections were performed using a 10 μL Hamilton microsyringe (Hamilton Company, Reno, Nev., USA) at a point 2 mm caudal and 2 mm lateral from bregma. Compounds were injected at a depth of 3 mm in a volume of 5 μL. Intravenous (i.v.) injections were performed by restraining the mouse in a Plexiglas holder, dipping the tail for 10 seconds in 40° C. warm water to dilate the tail vein, and subsequent injection into the vein with a 30-gauge needle. All compounds were dissolved in distilled water (i.c.v. injections) or physiologic saline (i.v. injections).

Antinociception was assessed using the 55° C. warm water tail-flick test. The latency to the first sign of a rapid tail-flick was taken as the behavioral end-point. Each mouse was first tested for baseline latency by immersing its tail in the water and recording the time to response. Mice not responding within 5 seconds were excluded from further testing (average latency=2.1 seconds). Mice were then administered the test compound and tested for antinociception at 10, 20, 30, 45, 60, 90, 120 and 180 min postinjection. A maximum score was assigned (100%) to animals not responding within 10 seconds. Antinociception was calculated by the following formula: % antinociception=100·(test latency ) control latency)/(10 ) control latency). Dose-response lines were constructed at times of peak agonist effect, and analyzed by linear regression using FLASHCALC software (17). All A₅₀ values (95% confidence limits) shown were calculated from the linear portion of the dose-response curve. A minimum of three doses/curve and 8-10 mice were used at each dose level.

The binding affinities and antinociceptive potency of 1-5 are summarized in Table 1 below (values for morphine sulfate are included for comparison purposes). The binding affinities and receptor preferences of the 1-selective DAMGO derivatives (2-5) are similar to the parent compound (0.56-1.3 nM Ki values for MOR, selectivity ratios for MOR over DOR and KOR of >500 and 100, respectively).

TABLE 1

| | A₅₀ i.c.v. | | [³H]DAMGO | [³H]Naltrindole | w[³H]U69,593 | |
|---|---|---|---|---|---|---|
| | (pmol per | A₅₀ i.v. | (μ) | (δ) | (κ) | Ratio |
| Opioid | mouse) | (μmol/kg) | Kᵢ (nM) ± S.E.M. | | | μ:δ:κ |
| Morphine | 2.384 | 7.84 | 0.79 ± 0.12 | 290 ± 38 | 12 ± 1.3 | 1:370:15 |
| DAMGO 1 | 30 | 1.88 | 0.56 ± 0.006 | 990 ± 35 | 270 ± 9.3 | 1:1900:510 |
| 2 | 2.0 | 0.20 | 0.68 ± 0.02 | 600 ± 44 | 190 ± 9.3 | 1:880:280 |
| 3 | 2.0 | 0.27 | 1.30 ± 0.16 | 730 ± 66 | 160 ± 10 | 1:560:120 |
| 4 | 19 | 0.72 | 1.30 ± 0.09 | >5000 | 170 ± 2.7 | 1:3800:130 |
| 5 | 2.0 | 1.15 | 0.66 ± 0.05 | 1600 ± 129 | 350 ± 51 | 1:2400:530 |

Table 1: Antinociceptive potencies(i.c.v. and i.v. in mouse 55° C. tail-flick assay) and binding affinities for DAMGO and related analogs at MOR, DOR and KOR.

In general, the addition of more water-soluble groups in the address segment of DAMGO (1) increased the i.c.v. potency of the analog compounds (2, 3, and 5). Surprisingly, compound 4 was significantly less potent than the other three, with a calculated A₅₀ value similar to 1. This potency difference was attributed to a much lower affinity/efficacy at the DOR or some unique physiochemical property that affects receptor interactions (e.g. ability to interact with opioid heterodimers and homodimers). It should be noted that after i.c.v. administration this compound had the longest duration of action of any of the compounds tested (>180 min versus 90-120 min). In contrast, the duration of action of glycopeptide 4 after i.v. administration was similar to the other glycopeptides (AUC calculations and visual inspection of time-course plots.

Based on previously published studies (10,11,15) the i.v. antinociceptive potencies of enkephalin-based glycopeptides is largely determined by their ability to penetrate the BBB by transcytosis (18) which in turn depends on the biousian character of the drugs. One may consider two extremes that result in differential delivery of a peptide drug into the CNS.

First, the peptide binds tightly to biologic membranes and is effectively removed from the solution. Secondly, the peptide remains in aqueous solution, effectively preventing it from interacting with biologic membranes. Thus, the goal in producing glycopeptides that are capable of effective BBB penetration and receptor binding/activation, is to balance the degree of glycosylation, which effectively determines the amount of time the glycopeptide spends in contact with the endothelial membrane of the BBB, as well as other membranes that the glycopeptide is likely to encounter [e.g. the cell membrane in which the G-protein coupled receptor (GPCR) is embedded]. Affinity for the membrane is still required for effective binding to the GPCR (19), but a certain amount of ‘membrane hopping’ is required for effective drug transport. Thus, if one were to plot the BBB transport or antinociceptive A₅₀ values versus the membrane affinity, one would predict a U-shaped or V-shaped curve. Note that log P_{o/w}, or “size-based’ analyses (20) can be useful for prediction of passive diffusion, but not for the prediction of endocytotic events.

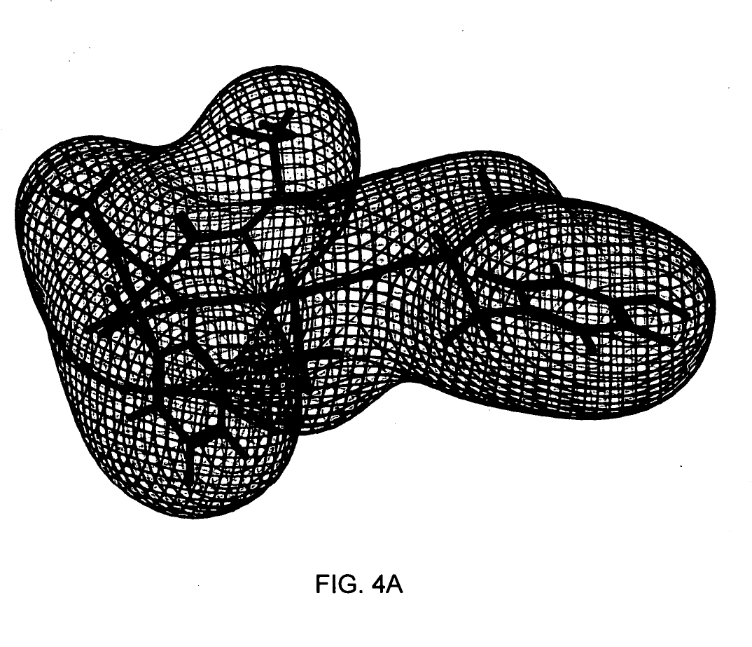

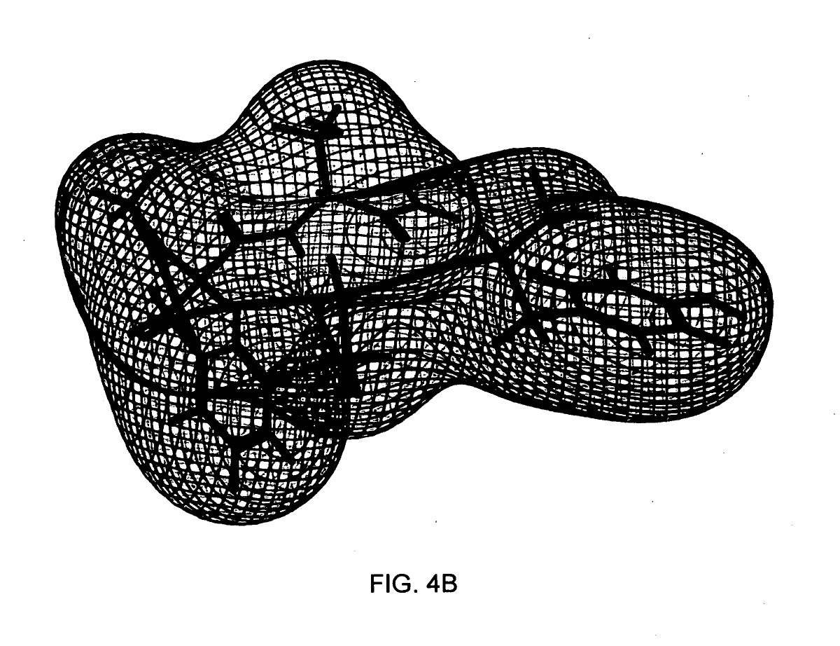

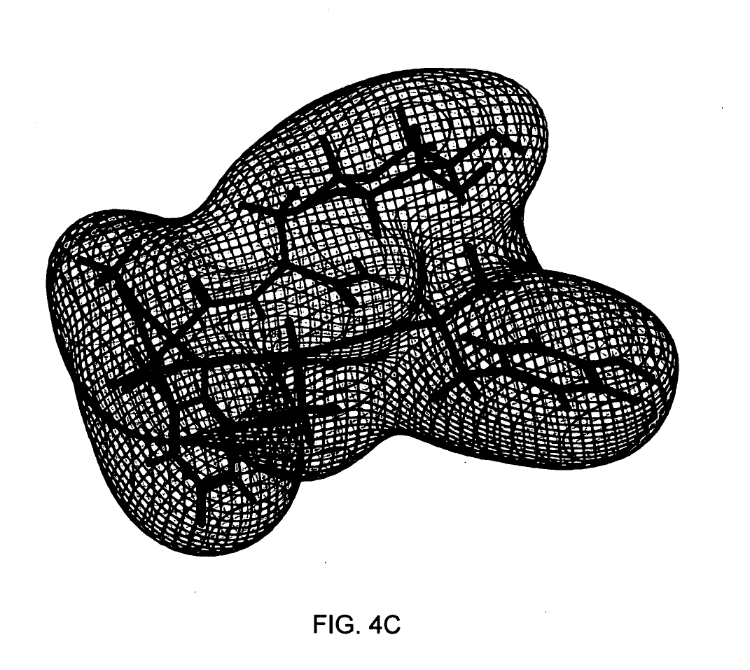

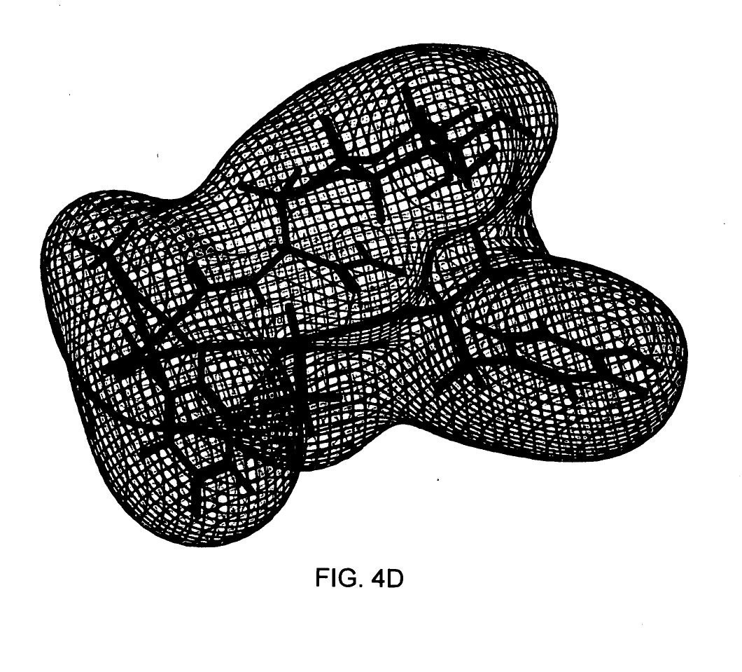

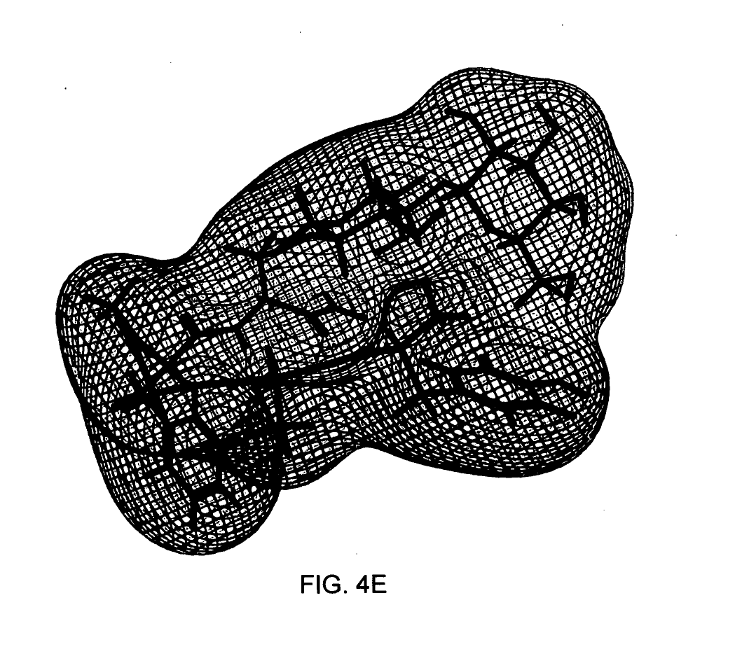

The amphipathic nature of DAMGO peptide analogs of compounds 2-5 can be visualized by calculation of a Connolly surface (solvent accessible surface) for each amphipathic species using the molecular mechanics package of MOE® (Chemical Computing Group, Montreal, QC, Canada) and by labeling the surface blue to indicate the lipophilic portion of the surface and red to indicate the hydrophilic portion of the surface (21; FIG. 4). The ratio of the two types of surface areas was used to create an expression of the amphipathicity using the formula A=^{-Awater/Alipid}. While the actual amphipathicity of each molecule will vary somewhat as each molecule is flexible and actually exists as an ensemble of conformations, it is not likely that the variation in A will be large. In any case, it is not likely that the order of amphipathicity will be different than what is predicted by this analysis.

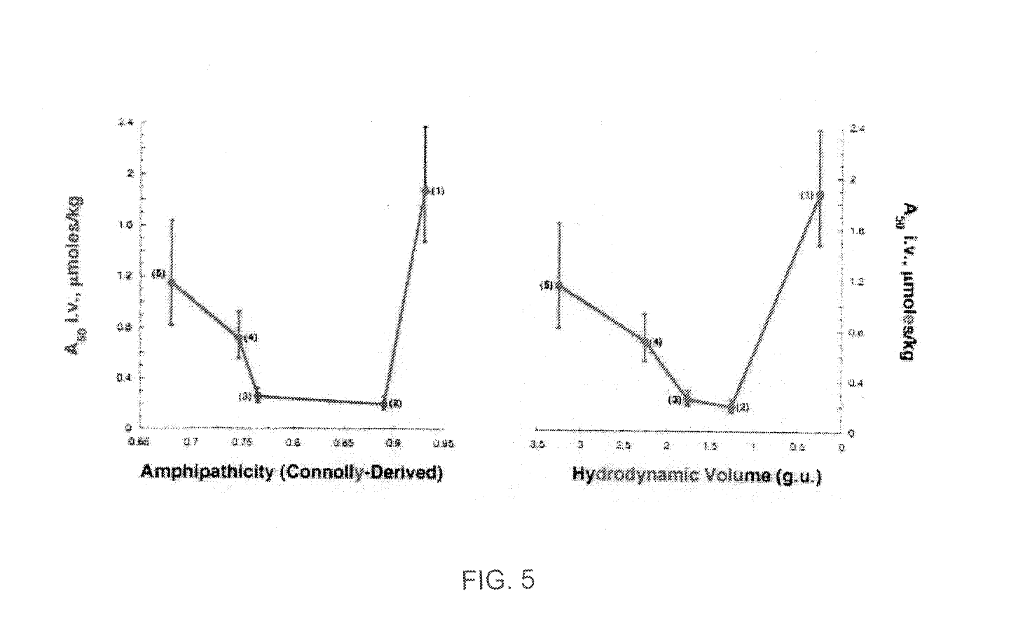

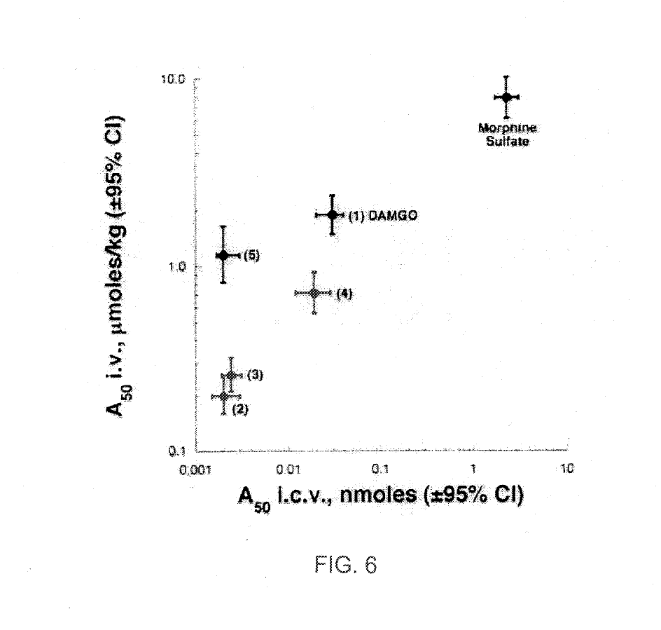

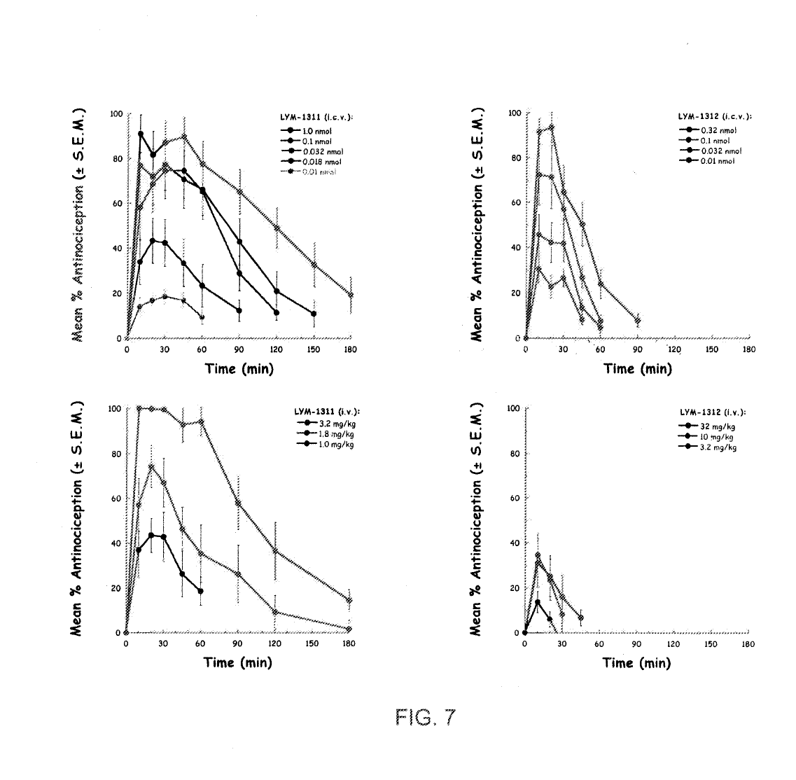

An alternative method of amphipathic analysis was also used. In this approach, hydrodynamic values of the hydrophilic portion of DAMGO (1) and DAMGO (2-5) were assigned, using accepted glucose unit values (g.u.; 22,23). For compounds 1 and 2 the single primary HO-group was assigned a value of 0.25 g.u. For compounds 2-5 the carboxamide group (C-terminal amide) was assigned a value of 1.00 g.u. This seems appropriate as the hydrodynamic change from Glc to GlcNAc or Gal to GalNAc is 1.00-2.00 g.u. Both methods of analysis are compared side-by-side (FIG. 5), and are plotted versus the i.v. A₅₀ values (with 95% confidence intervals). A plot of the i.c.v. antinociception vs. the i.v. antinociception leads to a non-linear plot (FIG. 6).

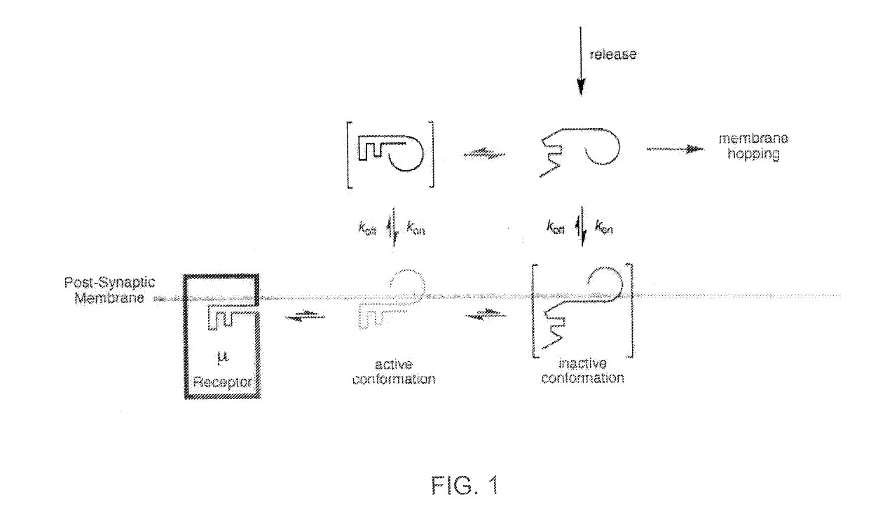

The inventors have found that μ-agonists based on the modified enkephalin analog DAMGO (1) exhibit properties consistent with those predicted by the ‘biousian hypothesis’ (10). Opioid neurotransmitters and most peptide-based opioid agonists that have been studied to date are amphipathic and bind tightly to model membranes and (presumably) to biologic membranes in vivo (FIG. 1). By incorporation of water-soluble ‘address segments’ into the C-terminus of neurotransmitter-like opioid agonist DAMGO (1) to produce peptide 2 and glycopeptide 3, corresponding increases in the bioavailability of the agonist ‘message segment’ is observed in vivo as indicated by increases in centrally mediated antinociception.

This is attributed in part to the increased water solubility of the neurotransmitter, which effectively increases the range of action of 1 from a few hundred Å, i.e. the distance across the synaptic cleft, to much larger distances, effectively allowing the peptide message in 2 and 3 to ‘hop’ from membrane surface to membrane surface, and thereby acquire drug-like properties. As the water solubility is further increased (glycopeptides 4 and 5), it is suggested that the affinity for the membrane is reduced to the point that interaction with the BBB is reduced (24), effectively reducing the CNS penetration of 4 and 5 and reducing the i.v. antinociceptive potency.

A number of factors determine the activity of a peptide-based drug candidate in the CNS. A primary factor is the bioavailability of the drug to the receptor populations of interest (25). The biousian behavior of the glycopeptides contributes to the observed potency differences of 1-5 via the ability of the molecules to associate with membranes both within the CNS and the periphery. The biousian behavior of the peptides of the invention can be attributed to the increased transport of the glycopeptides across the BBB (10) which is consistent with the inventors' previous findings with enkephalin-based glycopeptides with mixed 1/d activity.

Direct transport measurements of peptides or peptide analogs 1-5 can be used to further characterize the membrane-hopping ability of these peptides within the synaptic cleft and elsewhere. The specific in vivo antinociceptive potency of these molecules may be measured by determining the stability and physiological distribution of DAMGO analogs, such as compounds 1-5.

### REFERENCES

1. Lord J. A. H., Waterfield A. A., Hughes J., Kosterlitz H. W. (1977) Endogenous opioid peptides—multiple agonists and receptors. Nature;267:495-499.
2. Snyder S. H., Pasternak G. W. (2003) Historical review: opioid receptors. TIPS;24:198-205.
3. Mitchell S. A., Pratt M. R., Hruby V. J., Polt R. (2001) Solid-phase synthesis of O-linked glycopeptide analogues of enkephalin. J Org Chem;66:2327-2342 (vidi penitus).
4. Schiller P. W., Nguyen T. M. D., Weltrowska G., Wilkes B. C., Marsden B. J., Lemieux C., Chung N. N. (1992) Differential stereochemical requirements of mu vs delta-opioid receptors for ligand-binding and signal transduction—development of a class of potent and highly delta-selective peptide antagonists. Proc Natl Acad Sci USA;89:11871-11875.
5. Greene D. L., Hau V. S., Abbruscato T. J., Bartosz H., Misicka A., Lipkowski A. W., Hom S., Gillespie T. J., Hruby V. J., Davis T. P. Chem Biol Drug Des 2007; 69: 41-47 (1996) Enkephalin analog prodrugs: assessment of in vitro conversion enzyme cleavage characterization and blood-brain barrier permeability. J Pharm Exp Ther;277:1366-1375.
6. Begley D. J. (1996) The blood-brain barrier: principles for targeting peptides and drugs to the central nervous system. J Pharm Pharmacol;48:136-146.
7. Watkins K. J. (2001) Peptides: a boom in the making. Chem Eng News;January 8 issue:11-15.
8. Hawkins B. T., Davis T. P. (2006) The blood-brain barrier/neurovascular unit in health and disease. Pharm Rev;57:173-185.
9. Owens J., Lipinski C. (2003) Chris Lipinski discusses life and chemistry after the rule of five. Drug Discov. Today; 8:12-16.
10. Egleton R. D., Bilsky E. J., Tollin G., Muthu D., John Lowery Alves I., Davis P., Porreca F., Yamamura H. I., Yeomans L., Keyari C. M., Polt R. (2005) Biousian glycopeptides penetrate the blood-brain barrier. Tetrahedron Asymmetry; 16:65-75.
11. Muthu D., Polt R. (2005) New prospects for glycopeptide based analgesia: glycoside-induced penetration of the blood-brain barrier. Curr Drug Deliv; 2:59-73.
12. Deaton K. R., Feyen E. A., Nkulabi H. J., Morris K. F. (2001) Pulsed-field gradient NMR study of sodium dodecyl sulfate micelle-peptide association. Magn Reson Chem; 39:276-282.
13. Chatterjee C., Majumder B., Mukhopadhyay C. (2004) Pulsed-field gradient and saturation transfer difference NMR study of enkephalins in the ganglioside GM1 micelle. J Phys Chem B; 108:7430-7436.
14. Deguchi Y., Miyakawa Y., Sakurada S., Naito Y., Morimoto K., Ohtsuki S., Hosoya K., Terasaki T. (2003) Blood-brain barrier transport of a novel 11-specific opioid peptide, H-Tyr-D-Arg-Pheb-Ala-OH(TAPA). J Neurochem;84:1154-1161.
15. Handa B. K., Land A. C., Lord J. A., Morgan B. A., Lance M. J., Smith C. F. (1981) Analogues of beta-LPH61-64 possessing selective agonist activity at mu-opiate receptors. Eur J Pharmacol; 70:531-540.
16. Sargent D. F., Schwyzer R. (1986) Membrane lipid phase as catalyst for peptide receptor interactions. Proc Natl Acad Sci USA; 83:5774-5778.
17. Tallarida R. J., Murray R. B. (1987) Manual of Pharmacological Calculations with Computer Programs. Springer-Verlag New York Inc., New York.
18. Egleton R. D., Mitchell S. A., Huber J. D., Palian M. M., Polt R., Davis T. P. (2001) Improved blood-brain barrier penetration and enhanced analgesia of an opioid peptide by glycosylation. J Pharm Exp Ther;299:967-972.
19. Castano M. A. R. B., Fernandes M. X. (2006) Lipid membrane-induced optimization for ligand-receptor docking: recent tools and insights for the ‘membrane catalysis’ model. Eur Biophys J; 35:92-103.
20. Buchwald P., Bodor N. (1998) Octanol-water partition of nonzwitterionic peptides: predictive power of a molecular size-based model. Proteins: Struct, Funct, Genet;30:86-99.
21. Kelder J., Grootenhuis P. D. J., Bayada D. M., Delbressine L. P. C., Ploemen J.-P. (1999) Polar molecular surface as a dominating determinant for oral absorption and brain penetration of drugs. Pharm Res;16:1514-1519.
22. Kobata A., Yamashita K., Takasaki S. (1987) BioGel P-4 column chromatography of oligosaccharides: effective size of oligosaccharides expressed in glucose units. Methods Enzymol; 138: 84-94.
23. Guile G. R., Rudd P. M., Wing D. R., Prime S. B., Dwek R. A. (1996) A rapid high-resolution high-performance liquid chromatographic method for separating glycan mixtures and analyzing oligosaccharide profiles. Anal Biochem;240:210-226.
24. Segrest J. P., Jones M. K., Mishra V. K. (2002) Experimental and computational studies of the interactions of amphipathic peptides with lipid surfaces. Curr. Top. Membr.; 52:397-435.
25. Martin I. (2004) Prediction of blood-brain barrier penetration: are we missing the point? Drug Discov. Today; 9:161-162. Chem Biol Drug Des 2007; 69: 41-47.

### INCORPORATION BY REFERENCE

Each document, patent, patent application or patent publication cited by or referred to in this disclosure is incorporated by reference in its entirety. However, no admission is made that any such reference constitutes prior art and the right to challenge the accuracy and pertinence of the cited documents is reserved. Specifically, U.S. Provisional Application Nos. 60/806,751 and 60/819,428, filed Jul. 7, 2006; U.S. Publication No. US2006/0148679 A1 and U.S. application Ser. No. 10/594,515, filed Sep. 28, 2006, and U.S. Provisional Application [as yet undesignated; “Glycopeptide Targets”] filed Jun. 22, 2007; are incorporated by reference.

1. A DAMGO ([D-Ala²,Me-Phe⁴Gly⁵(ol)]enkephalin) analog in which one or more positions in formula I is substituted with a hydrophilic group that increases the hydrophilicity of the DAMGO analog compared to DAMGO;
wherein DAMGO is represented by formula (I):

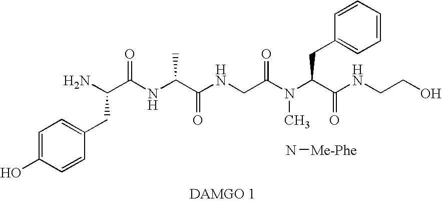

2. The DAMGO analog of claim 1, which comprises a hydrophilic moiety at its C-terminus other than unsubstituted glycinol.

3. The DAMGO analog of claim 1, wherein the hydroxy group on the glycinol moiety is substituted with a group more hydrophilic than hydroxy.

4. The DAMGO analog of claim 1, wherein glycinol is replaced with an amino acid residue or modified amino acid residue more hydrophilic than glycinol.

5. The DAMGO analog of claim 1, which comprises the following structure: 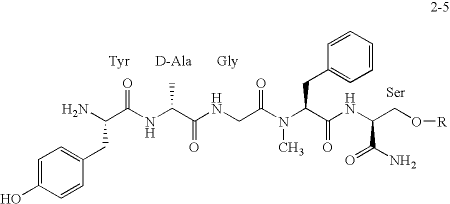
wherein R is hydrogen or a hydrophilic group.

6. The DAMGO analog of claim 4, wherein R is a sugar or carbohydrate group.

7. The DAMGO analog of claim 4, wherein R comprises a phosphate, pyrophosphate, phosphonate, sulfate, or sulfonate group, which groups may be further substituted with alkyl, cycloalkyl or aryl.

8. The DAMGO analog of claim 1, wherein the glycinol moiety is replaced by L-serine amide, L-serine amide β-D-xyloside, L-serine amide β-D-glucoside, or L-serine amide β-lactoside.

9. The DAMGO analog of claim 1 having one of the following structures:

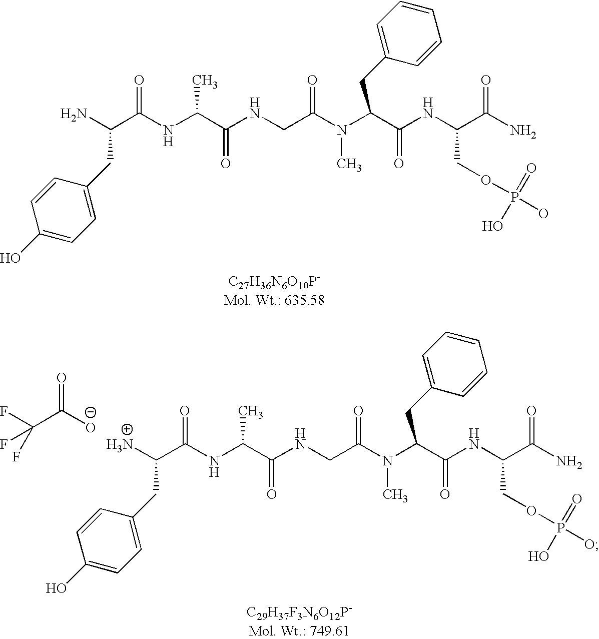

10. The DAMGO analog of claim 1 having one of the following structures:

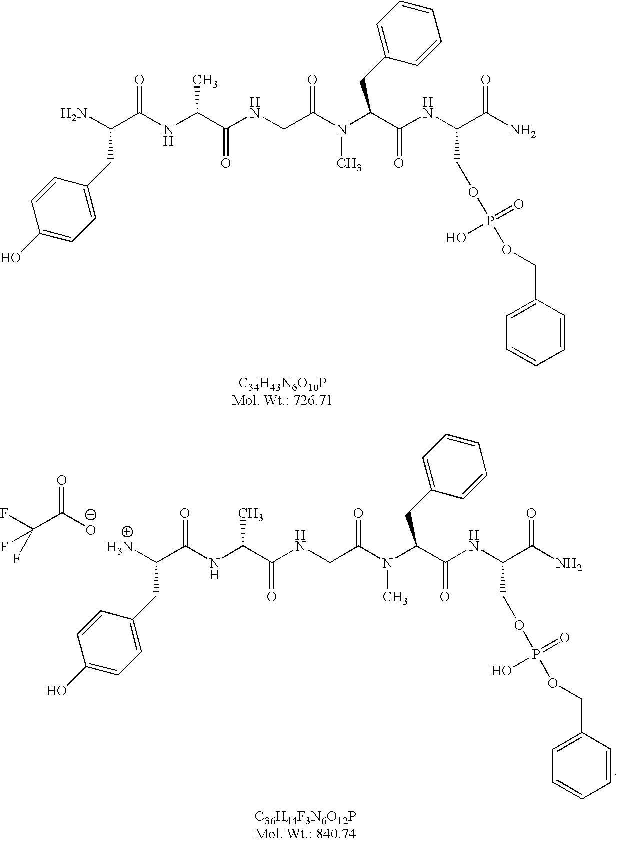

11. The DAMGO analog of claim 1 which is a mu opioid agonist, inverse agonist, or antagonist.

12. The DAMGO analog of claim 1 in which the degree of glycosylation ranges from 1.25 to 1.75 in terms of hydrodynamic glucose units or ranges between 0.75 to 0.90 g. u. in terms of surface-derived amphipathicity values.

13. The DAMGO analog of claim 1 which has a molecular mass of about 1,000 Da or less or which may contain up to 9 amino acid residues.

14. The DAMGO analog of claim 1 which has a lower binding affinity for the post-synaptic membrane than DAMGO.

15. The DAMGO analog of claim 1 which has a lower binding affinity for opioid mu receptor than DAMGO.

16. The DAMGO analog of claim 1 which penetrates the blood brain barrier faster or to a greater extent than DAMGO.

17. The DAMGO analog of claim 1 further comprising a modification to increase its biological half-life, biological adsorption, or passage across the blood brain barrier.

18. A pharmaceutical composition comprising the DAMGO analog of claim 1 and at least one pharmaceutically acceptable carrier or excipient.

19. A pharmaceutical composition comprising a liposome or lipid bilayer in which the DAMGO analog of claim 1 is present, wherein said DAMGO analog has been modified to incorporate into said liposome or lipid bilayer.

20. A method for relieving or modulating pain or for inducing analgesia or sedation comprising administering an effective amount of the DAMGO analog of claim 1 to a subject in need thereof.

21. A method for reducing blood pressure, respiration, or decreasing bowel motility comprising administering an effective amount of the DAMGO analog of claim 1 to a subject in need thereof.

22. A method for modulating itching, nausea, euphoria, or miosis (constricted pupils) comprising administering to a subject in need thereof an effective amount of the DAMGO analog of claim 1.

23. A method for treating a disease or disorder mediated by a mu opioid receptor comprising administering to a subject in need thereof an effective amount of the DAMGO analog of claim 1.

24. A method for identifying a DAMGO analog that binds to the mu opioid receptor and which exhibits biousian properties compared to DAMGO, comprising:
determining the ability of said analog to hop from one membrane surface to another, its ability to assume either a hydrophilic and hydrophobic configuration and/or its ability to freely diffuse throughout the aqueous compartments of a biologic organism,
selecting a DAMGO analog having a greater ability to hop from one membrane surface to another, a greater ability to assume both a hydrophilic and hydrophobic configuration, or a greater ability to freely diffuse throughout the aqueous compartments of the biologic organism, compared to DAMGO, and
selecting a DAMGO analog that binds to the mu opioid receptor.

25. The method of claim 24, wherein said DAMGO analog comprises: 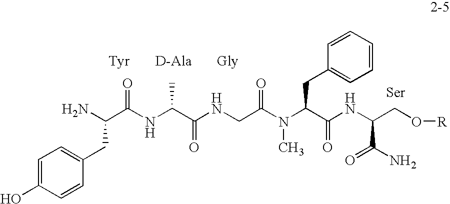
where R is H or a hydrophilic group.

26. The method of claim 24, wherein membrane hopping ability or its biousian property is determined by at least one method selected from the group consisting of reversed-phase HPLC retention times; Langmuir isotherms to lipophilic surfaces; data from surface plasmon resonance (SPR); vesicle binding using microcalorimetry; and binding to micelles and bicelles using nuclear magnetic resonance (NMR) or circular dichroism (CD).
